(19) United States
(12) Patent Application Publication
(10) Pub. No.: US 2025/0064007 A1
(43) Pub. Date: Feb. 27, 2025

(54) Self-Fertilization as Methods for Stabilizing Ploidy after Colchicine Treatment Model

(71) Applicant: Rev Genomics, Inc., Oakland, CA (US)

(72) Inventors: Phillip Adam Conklin, Oakland, CA (US); Bertrand R. Vick, Oakland, CA (US)

(21) Appl. No.: 18/811,676

(22) Filed: Aug. 21, 2024

Related U.S. Application Data
(60) Provisional application No. 63/534,304, filed on Aug. 23, 2023.

(51) Int. Cl. A01H 1/08; A01H 6/28; A01N 33/06

(52) U.S. Cl. CPC A01H 1/08; A01H 6/28; A01N 33/06

(57) ABSTRACT

Exemplary methods include a method of stabilizing ploidy including doubling a plant's ploidy, self-fertilizing the doubled plant, and creating an offspring with a germline ploidy of a desired doubled ploidy targeted when doubling the plant's ploidy. Another exemplary method of stabilizing ploidy includes self-fertilizing a haploid plant, and creating an offspring plant with a germline ploidy that is diploid. A further method of stabilizing ploidy includes treating a female microtubule inhibitor treated plant with an ethylene blocker, producing male reproductive organs and pollen by the female microtubule inhibitor treated plant, fertilizing a clone of the female microtubule inhibitor treated plant with the pollen, and producing seeds and/or offspring.

### CROSS REFERENCE TO RELATED APPLICATIONS

The present application claims the priority benefit of U.S. Provisional Patent Application Ser. No. 63/534,303 filed on Aug. 23, 2023, and titled, “Self-Fertilization as Methods for Stabilizing Ploidy After Colchicine Treatment Model,” which is hereby incorporated by reference in its entirety, including any and all appendices.

### BRIEF SUMMARY OF EXEMPLARY EMBODIMENTS

Exemplary methods include a method of stabilizing ploidy including doubling a plant's ploidy, self-fertilizing the doubled plant, and creating an offspring with a germline ploidy of a desired doubled ploidy targeted when doubling the plant's ploidy. Another exemplary method of stabilizing ploidy includes self-fertilizing a haploid plant, and creating an offspring plant with a germline ploidy that is diploid. A further method of stabilizing ploidy includes treating a female microtubule inhibitor treated plant with an ethylene blocker, producing male reproductive organs and pollen by the female microtubule inhibitor treated plant, fertilizing a clone of the female microtubule inhibitor treated plant with the pollen, and producing seeds and/or offspring.

In various exemplary embodiments, the plant may be from a Cannabis genus, including marijuana and hemp. Some exemplary embodiments may include fertilizing the female microtubule inhibitor treated plant itself with the pollen it produces. Various exemplary embodiments may include identifying a plant with spontaneously doubled haploids.

A leaf ploidy, according to various exemplary embodiments, may be approximately 52.69% diploid and approximately 11.83% tetraploid, or approximately 51.46% diploid and approximately 10.26% tetraploid, or approximately 38.37% diploid and approximately 7.69% tetraploid.

Certain exemplary embodiments may include the offspring plant with the germline ploidy being 100% diploid.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. With respect to the color drawings, they are necessary as the only practical medium by which aspects of the claimed subject matter (including data) may be accurately conveyed. The color of the Cannabis leaves may vary depending upon the circumstances and/or cultivar.

The accompanying drawings, where like reference numerals refer to identical or functionally similar elements throughout the separate views, together with the detailed description below, are incorporated in and form part of the specification, and serve to further illustrate embodiments of concepts that include the claimed disclosure and explain various principles and advantages of those embodiments.

FIG. 1A shows leaves from the cultivar “Standing Liberty” either (left) in a haploid state, or (middle) chimeric/mixaploid state, and (right) from the diploid parent of the haploid.

FIG. 1B shows the flow cytometer data from the cultivar “Standing Liberty” in a haploid state.

FIG. 1C shows the flow cytometer data from the cultivar “Standing Liberty” in a chimeric/mixaploid state.

FIG. 1D shows the flow cytometer data from the cultivar “Standing Liberty” from the diploid parent of the haploid.

FIG. 2A shows leaves from the cultivar “Herbaceous Sting” either (left) in a haploid state, or (middle) chimeric/mixaploid state, and (right) from the diploid parent of the haploid.

FIG. 2B shows the flow cytometer data from the cultivar “Herbaceous Sting” in a haploid state.

FIG. 2C shows the flow cytometer data from the cultivar “Herbaceous Sting” in a chimeric/mixaploid state.

FIG. 2D shows the flow cytometer data from the cultivar “Herbaceous Sting” from the diploid parent of the haploid.

FIG. 3A shows leaves from the cultivar “Operation Green Harvest Haze” either (left) in a haploid state, or (middle) chimeric/mixaploid state, and (right) from the diploid parent of the haploid.

FIG. 3B shows the flow cytometer data from the cultivar “Operation Green Harvest Haze” in a haploid state.

FIG. 3C shows the flow cytometer data from the cultivar “Operation Green Harvest Haze” in a chimeric/mixaploid state.

FIG. 3D shows the flow cytometer data from the cultivar “Operation Green Harvest Haze” from the diploid parent of the haploid.

FIG. 4A shows leaves from the cultivar “Cryptic Lineage Legacy” either (left) in a haploid state, or (middle) chimeric/mixaploid state, and (right) from the diploid parent of the haploid.

FIG. 4B shows the flow cytometer data from the cultivar “Cryptic Lineage Legacy” in a haploid state.

FIG. 4C shows the flow cytometer data from the cultivar “Cryptic Lineage Legacy” either in a chimeric/mixaploid state.

FIG. 4D shows the flow cytometer data from the cultivar “Cryptic Lineage Legacy” from the diploid parent of the haploid.

FIG. 5A shows leaves from the (left) self-fertilized doubled haploid cultivar “Mary Jane Manhunt” in a diploid (2n) state, or (middle) haploid (n) state, and (right) from the diploid (2n) parent of the haploid. The mixaploid data for the colchicine treated haploid was not gathered.

FIG. 5B shows the flow cytometer data from the self-fertilized doubled haploid cultivar “Mary Jane Manhunt” in a diploid (2n) state.

FIG. 5C shows the flow cytometer data from the haploid (n) state.

FIG. 5D shows the flow cytometer data from diploid (2n) parent of the haploid.

FIG. 6 shows one exemplary method of stabilizing ploidy.

FIG. 7 shows another exemplary method of stabilizing ploidy.

### DETAILED DESCRIPTION OF EXAMPLE EMBODIMENTS

The Cannabis plant is unique in that it is rarely able to spontaneously convert haploid genomes (In) to diploid genomes (2n) when haploidy has been induced by artificial means. This rare characteristic of the Cannabis plant presents challenges when attempting to produce so-called “doubled haploids,” which have been shown to be valuable tools in plant breeding programs. Specifically, doubled haploid plants of a given species may be mated to one another to produce F1-hybrid seeds that give rise to identical plants. Consistency across individuals of a crop is a valuable agronomic trait—something that is non-existent in Cannabis crops cultivated from seed.

Haploid plants can be treated with colchicine or other chemicals (any chemical that will achieve increasing a ploidy level such as a doubling of the chromosomes with Oryzalin) to double the ploidy level to achieve a more stable and useful diploid (2n) state. In some exemplary embodiments, a colchicine concentration (in water) of 0.25% may be used in addition to 0.5%, 1% and 5%. An issue arises with Cannabis in that not all the cells or tissues of a colchicine treated plant will double their ploidy. Colchicine treated haploid Cannabis plants very rarely, if ever, achieve a fully diploid state.

On the single-cellular level, colchicine treatment can arrest mitosis just before anaphase by destabilizing microtubules, which are normally required to separate the doubled chromosomes synthesized during S-phase. Depending on the local dose, the cell either completes mitosis and gives rise to two cells with non-doubled chromosomes, or incomplete mitosis occurs, resulting in one cell with doubled chromosomes. If the entire plant can be derived from the latter single doubled event, the basal ploidy will be uniform throughout the plant.

On the multi-cellular level, doubling the ploidy of plants requires colchicine treatment applied to the shoot apical meristem (SAM). The SAM is composed of three distinct cell layers: L1 (outermost layer), L2 (middle layer), and L3 (innermost layer). Within the SAM, stem cell division takes place along the peripheral zone where new leaves emerge (Heidstra, 2014). The L1 gives rise to the epidermis or the protective covering of the leaf and stem, L2 to the sub epidermal photosynthetic mesophyll tissue, and L3 to the vascular tissue including the xylem and phloem. L1 and L2 divide anticlinally or parallel to each other and thus remain distinct layers (Meyerowitz, 1997). On occasion, a cell in the SAM can divide periclinally whereby the daughter cell invades and acquires the identity one layer out and brings its ploidy with it. This organizational dynamic creates a stable yet mutable developmental path. In fact, the discovery of the stability of different layers within a meristem was discovered by creating a chimera using colchicine on Datura. The researchers were able to follow the developmental lineage of the differently sized cells due to the different ploidy levels within the SAM (Satina, 1940).

Aside from a scenario in which one ploidy has a growth advantage over another, to double the entire plant completely and thoroughly, each layer of the SAM would need an independent doubling event. Doubling the ploidy with uniformity of the entire SAM is unlikely due to the complexity of the SAM's multicellular three-dimensional dome-like structure and the requirement for doubling events in each layer of every cell column. Indeed, several report mixaploid or chimeric plants after colchicine treatment (Sakhanokho, 2004).

The fact that Cannabis is very stable in a haploid state (the inventors have 12 independent exemplary embodiments of stable haploidy in Cannabis) suggests diploid tissue may have little to no competitive growth advantage over haploid tissue, which means they can coexist. For example, if a successful conversion of both the L1 and L2 layer to a diploid state but the L3 layer remains haploid after colchicine treatment, L3 layer may unpredictably contribute haploid cells to the L2 layer and if this happens in the anther it could negatively affect gamete development. These unstable scenarios are not practical for a commercial seed producer that requires consistency.

According to exemplary embodiments, colchicine treated Cannabis plants can be “selfed” to produce seeds. Self-fertilizing a doubled haploid will form a diploid zygote from male and female gametes, each contributing identical haploid chromosome content. Because this single celled zygote will give rise to the entire plant (including all three layers of the SAM) seeds give rise to stable plants with uniform basal ploidy (De Rybel, 2016). Alternative methods of producing diploid plants from haploid plants require laborious analytical methods to ensure that all cells and tissue layers are fully diploid. Our method for establishing diploidy through self-fertilization is an elegant alternative to previous methods.

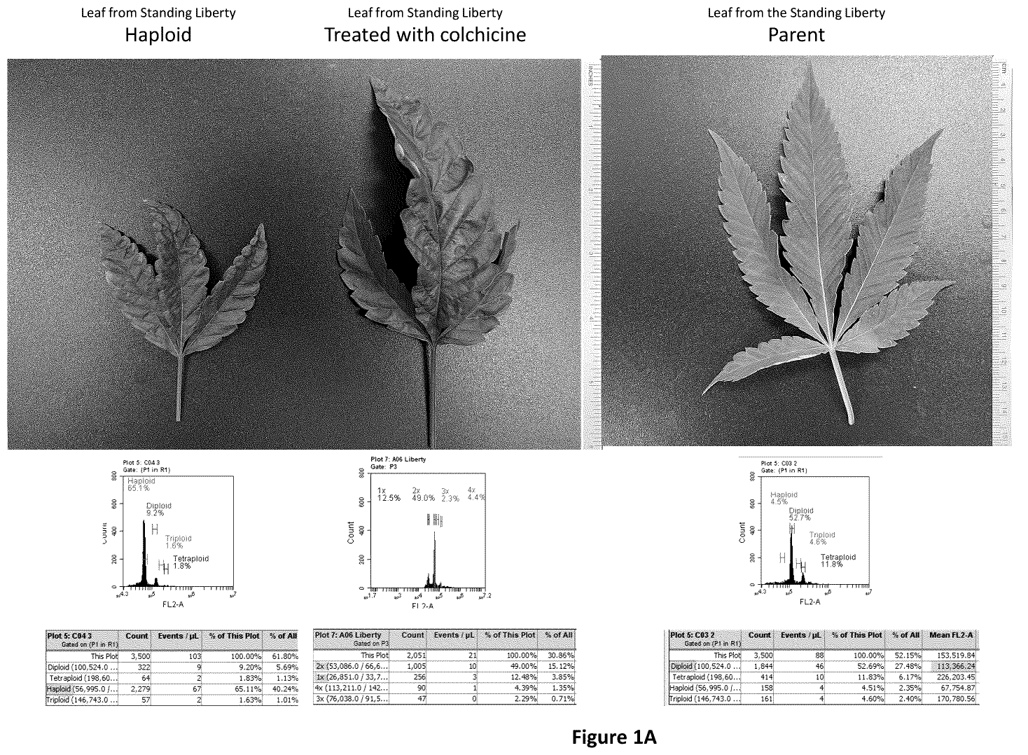

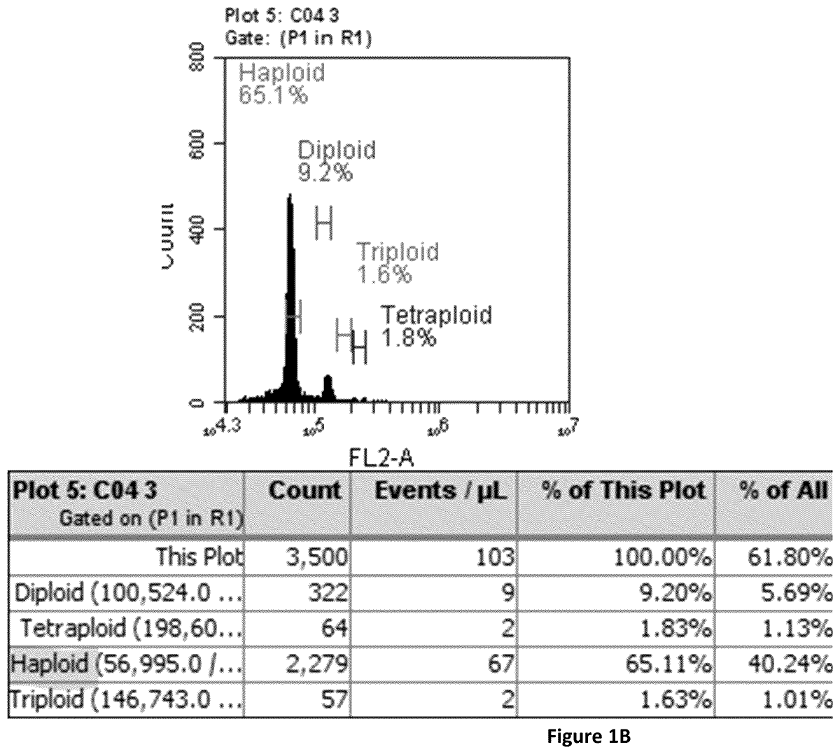

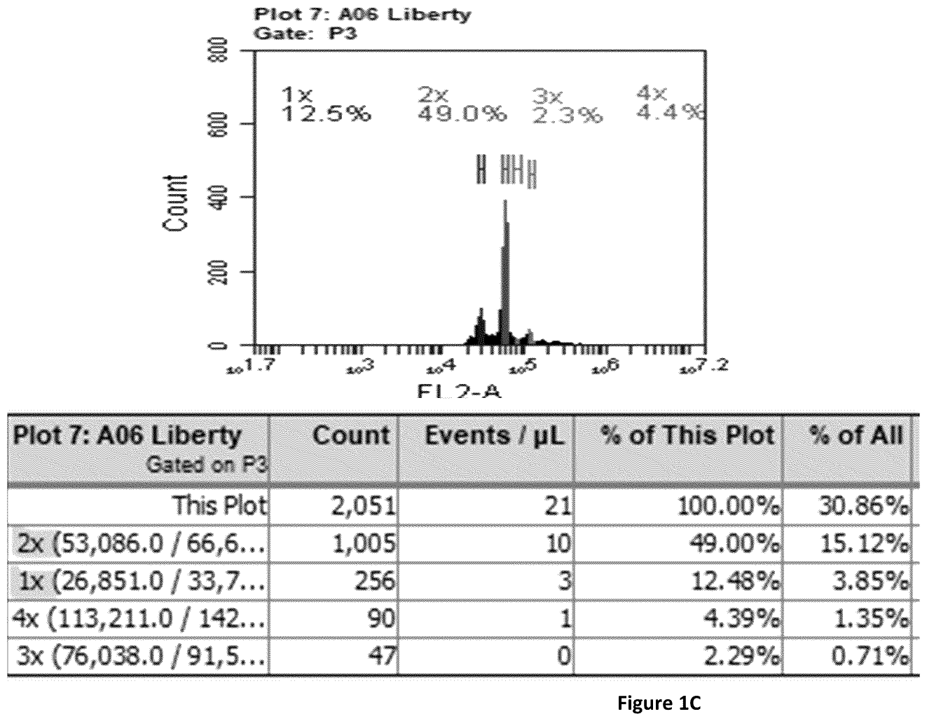

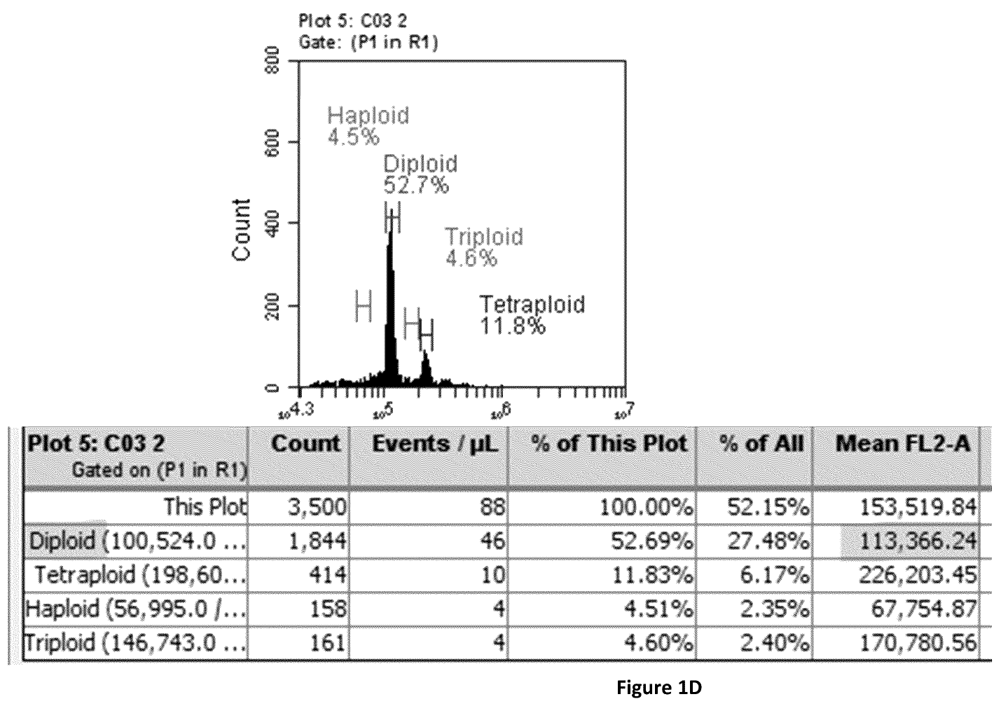

FIG. 1A shows exemplary leaves from the cultivar “Standing Liberty” either (left) in a haploid (n) state, or (middle) mixaploid (n+2n) state, and (right) from the diploid (2n) parent of the haploid. A “mixaploid state” refers to a condition in which a plant or organism has cells with varying ploidy levels, meaning some cells have different numbers of chromosome sets.

A “mixaploid state” refers to a condition in which a plant or organism has cells with varying ploidy levels, meaning some cells have different numbers of chromosome sets.

### Understanding Ploidy Levels:

Haploid (n): Cells with a single set of chromosomes.

Diploid (2n): Cells with two sets of chromosomes, typical of most organisms, including humans.
Polyploid: Cells with more than two sets of chromosomes. Examples include:
Triploid (3n): Three sets of chromosomes.
Tetraploid (4n): Four sets of chromosomes.
Hexaploid (6n): Six sets of chromosomes.
Mixaploidy: The condition of having different ploidy levels within the same organism. This can occur naturally or be induced artificially. It might result from irregular cell division, somatic mutation, or experimental treatments.

### Possible Scenarios of Mixaploidy:

Somatic Mosaicism: An organism may have cells with different ploidy levels due to endoreduplication during development. This results in a mosaic pattern where some cells are diploid while others are polyploid.

Chimeras: An organism composed of genetically distinct cells. For example, grafting can result in a plant with tissues that have different ploidy levels.

Experimental Induction: Researchers might create mixaploid conditions to study the effects of different ploidy levels on plant development and physiology.

FIG. 1B shows exemplary flow cytometer data from the cultivar “Standing Liberty” in a haploid (n) state, in which approximately 65.11% of the cells counted are haploid, approximately 9.2% are diploid. The diploid cells are likely a result of naturally occurring endoreduplication or spontaneous doubling, whereas the triploid and tetraploid cell counts do not have a distinct peak apart from the background noise.

FIG. 1C shows an exemplary mixaploid (n+2n) state plot shows a ploidy of approximately 49% diploid, approximately 12.48% haploid, and approximately 4.39% tetraploid. The mixaploid state has a pattern distinct from the haploid and diploid, which show mostly diploid cells, which likely accounts for a mostly successful doubling, but also a stubbornly stable subset of haploid cells, which do have their own distinct peak.

FIG. 1D shows an exemplary diploid (2n) parent of the haploid shows approximately 52.69% diploid, approximately 11.83% tetraploid, which is most likely from naturally occurring endoreduplication. This diploid parent also has counts from haploid and triploid cells that do not have their own distinct peak apart from the noise.

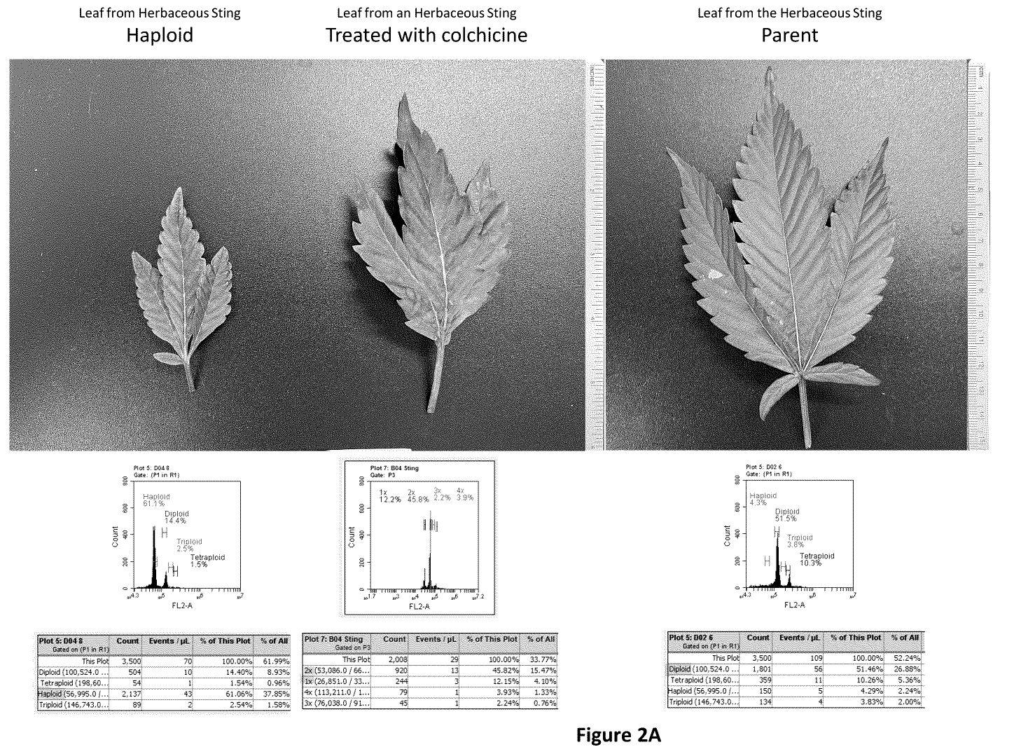

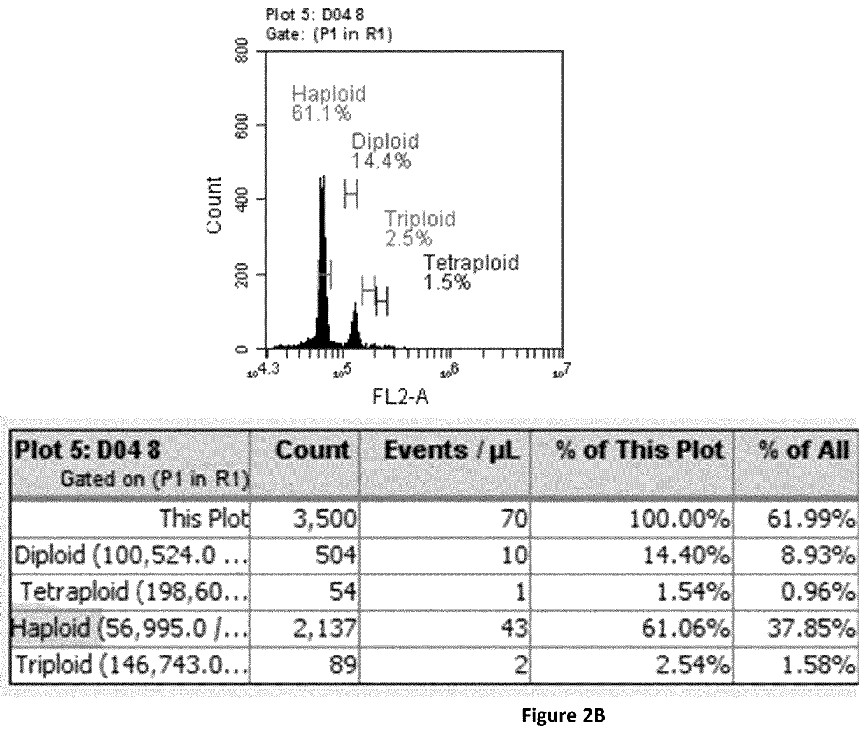

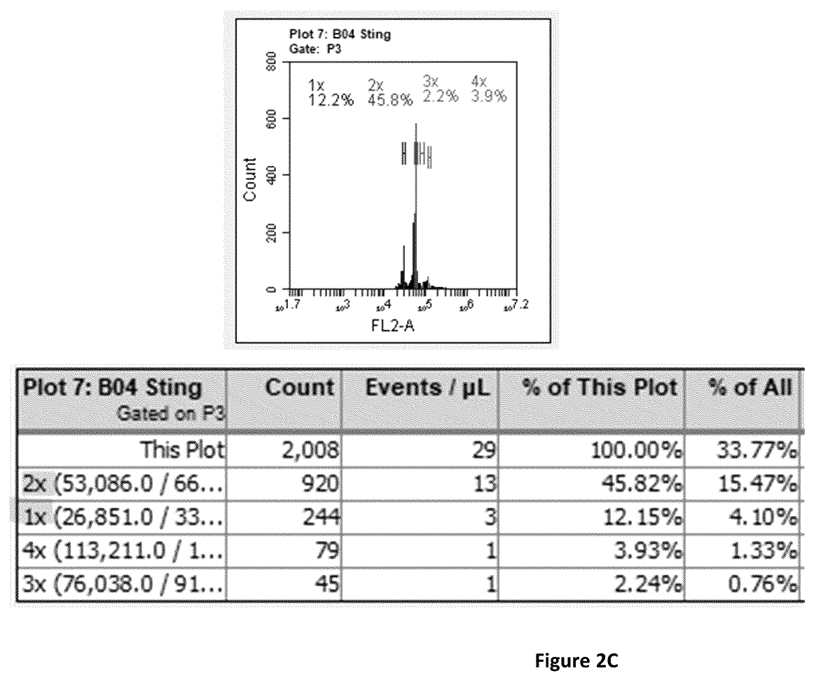

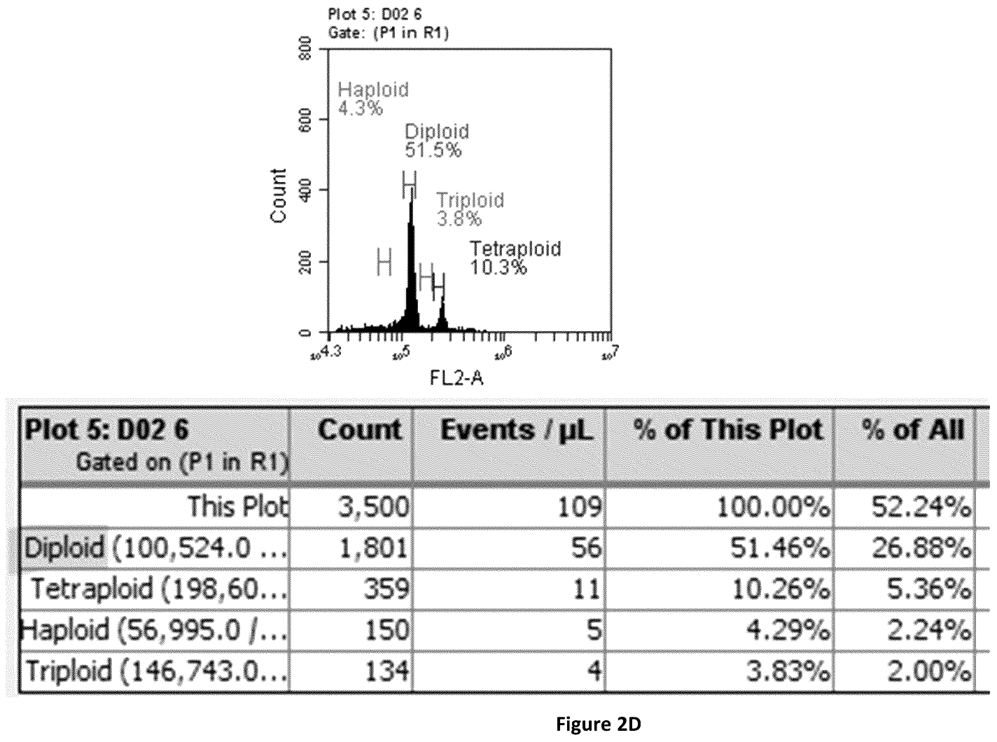

FIG. 2A shows exemplary leaves from the cultivar “Herbaceous Sting” either (left) in a haploid (n) state, or (middle) mixaploid (n+2n) state, and (right) from the diploid (2n) parent of the haploid. A “mixaploid state” refers to a condition in which a plant or organism has cells with varying ploidy levels, meaning some cells have different numbers of chromosome sets.

FIG. 2B shows exemplary flow cytometer data from the cultivar “Herbaceous Sting” in a haploid (n) state, in which approximately 61.06% of the cells counted are haploid, approximately 14.4% are diploid. The diploid cells are likely a result of naturally occurring endoreduplication or spontaneous doubling, whereas the triploid and tetraploid cell counts do not have a distinct peak apart from the background noise.

FIG. 2C shows an exemplary mixaploid (n+2n) state plot shows a ploidy of approximately 45.82% diploid, approximately 12.15% haploid, and approximately 3.93% tetraploid The mixaploid state has a pattern distinct from the haploid and diploid, which show mostly diploid cells, which likely accounts for a mostly successful doubling, but also a stubbornly stable subset of haploid cells, which do have their own distinct peak.

FIG. 2D shows an exemplary diploid (2n) parent of the haploid shows approximately 51.46% diploid, approximately 10.26% tetraploid, which is most likely from naturally occurring endoreduplication. This diploid parent also has counts from haploid and triploid cells that do not have their own distinct peak apart from the noise.

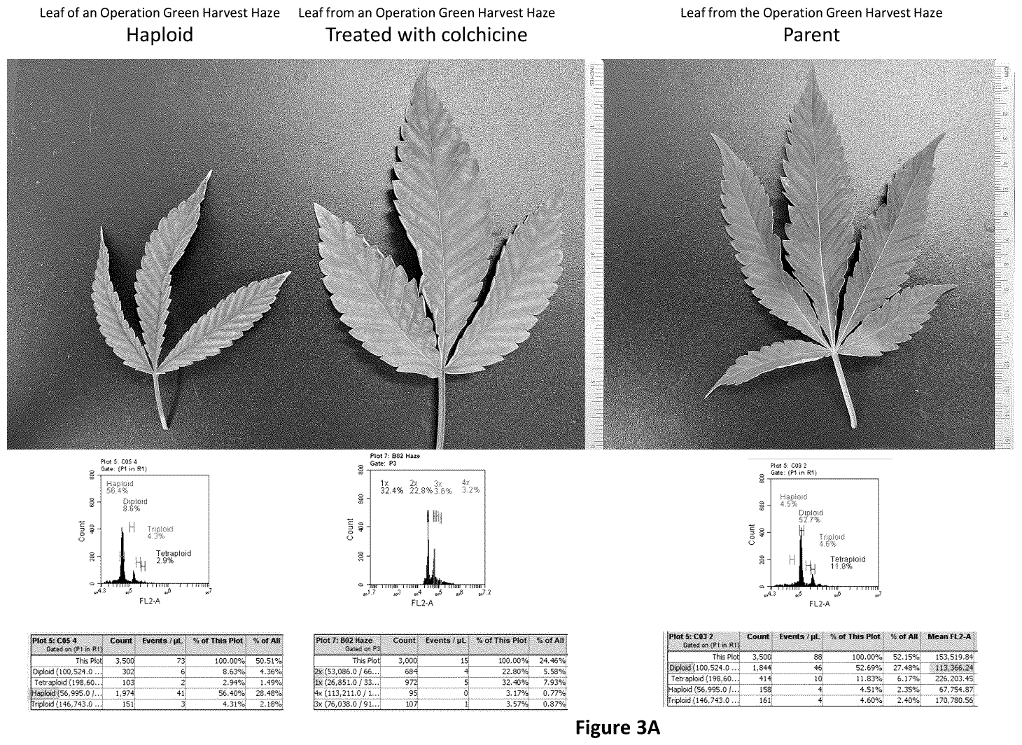

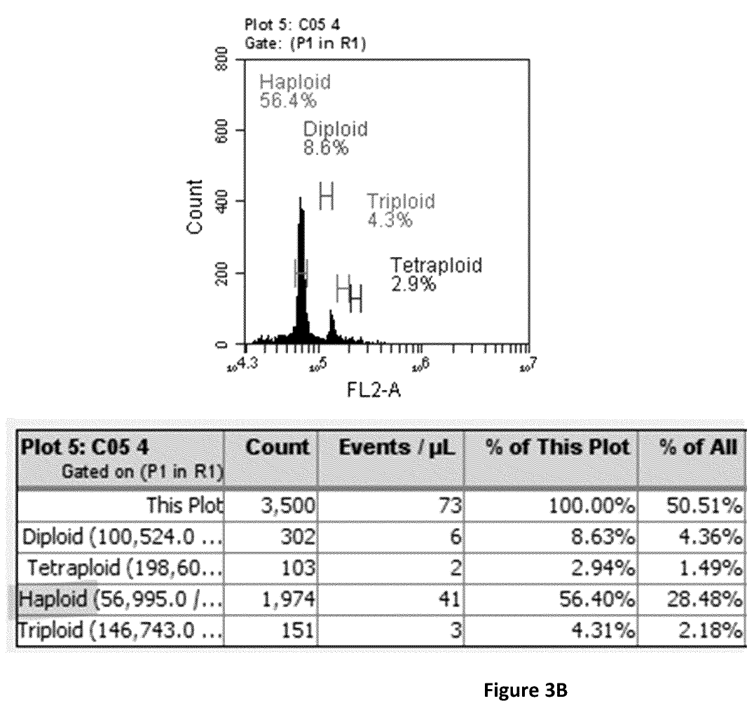

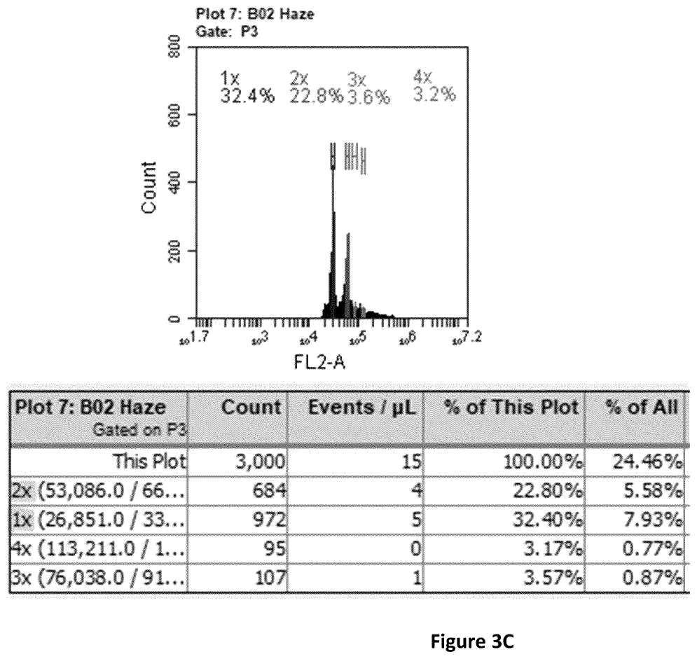

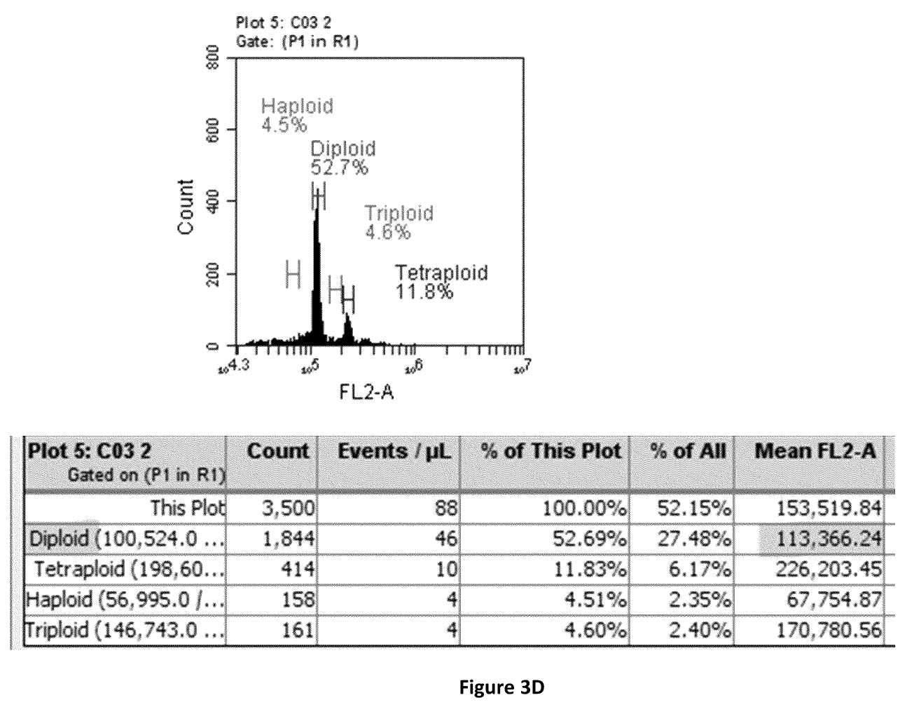

FIG. 3A shows exemplary leaves from the cultivar “Operation Green Harvest Haze” either (left) in a haploid (n) state, or (middle) mixaploid (n+2n) state, and (right) from the diploid (2n) parent of the haploid. A “mixaploid state” refers to a condition in which a plant or organism has cells with varying ploidy levels, meaning some cells have different numbers of chromosome sets.

FIG. 3B shows exemplary flow cytometer data from the cultivar “Operation Green Harvest Haze” in a haploid (n) state, in which approximately 56.4% of the cells counted are haploid, approximately 8.63% are diploid. The diploid cells are likely a result of naturally occurring endoreduplication or spontaneous doubling, whereas the triploid and tetraploid cell counts do not have a distinct peak apart from the background noise.

FIG. 3C shows exemplary mixaploid (n+2n) state plot shows a ploidy of approximately 22.8% diploid, approximately 32.4% haploid, and approximately 3.1% tetraploid. The mixaploid state has a pattern distinct from the haploid and diploid, which show mostly diploid cells, which likely accounts for a mostly successful doubling, but also a stubbornly stable subset of haploid cells, which do have their own distinct peak.

FIG. 3D shows an exemplary diploid (2n) parent of the haploid shows approximately 52.69% diploid, approximately 11.83% tetraploid, which is most likely from naturally occurring endoreduplication. This diploid parent also has counts from haploid and triploid cells that do not have their own distinct peak apart from the noise.

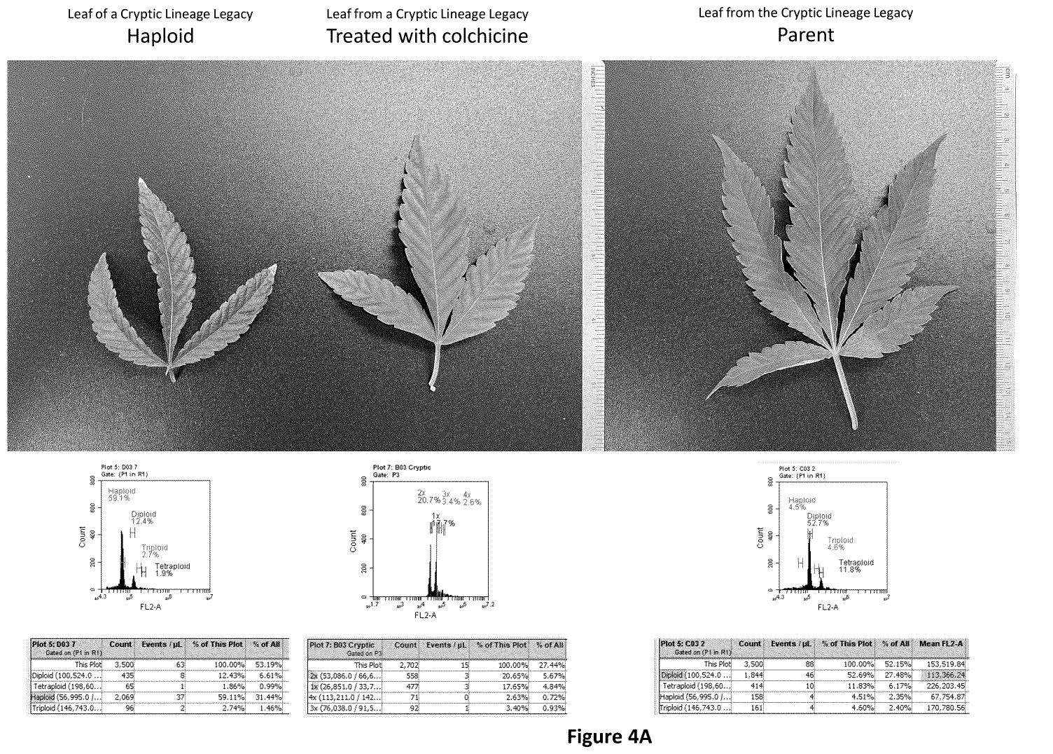

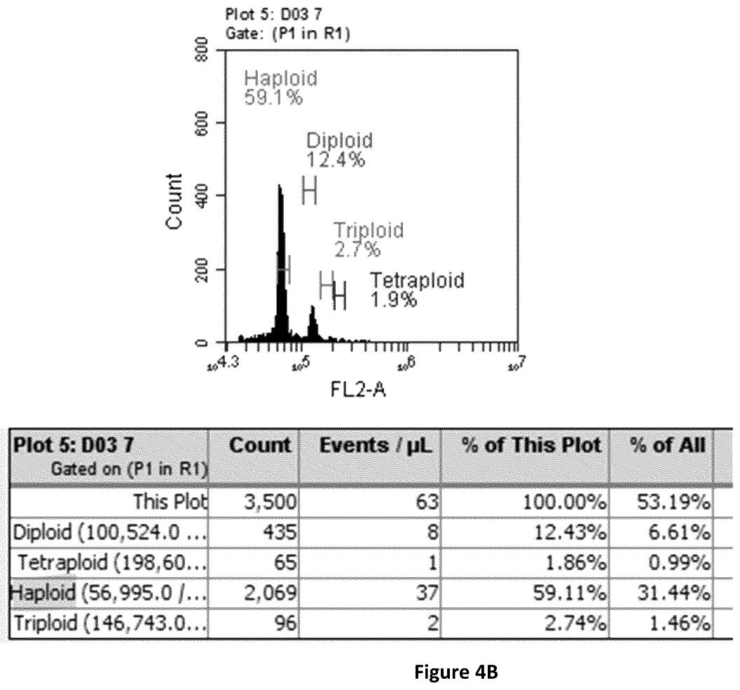

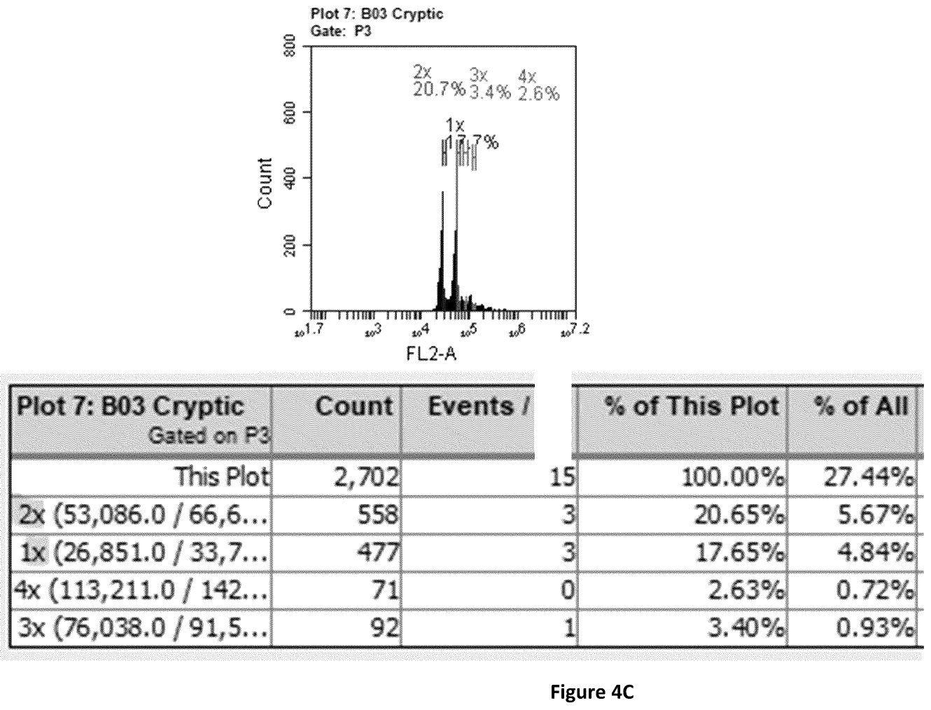

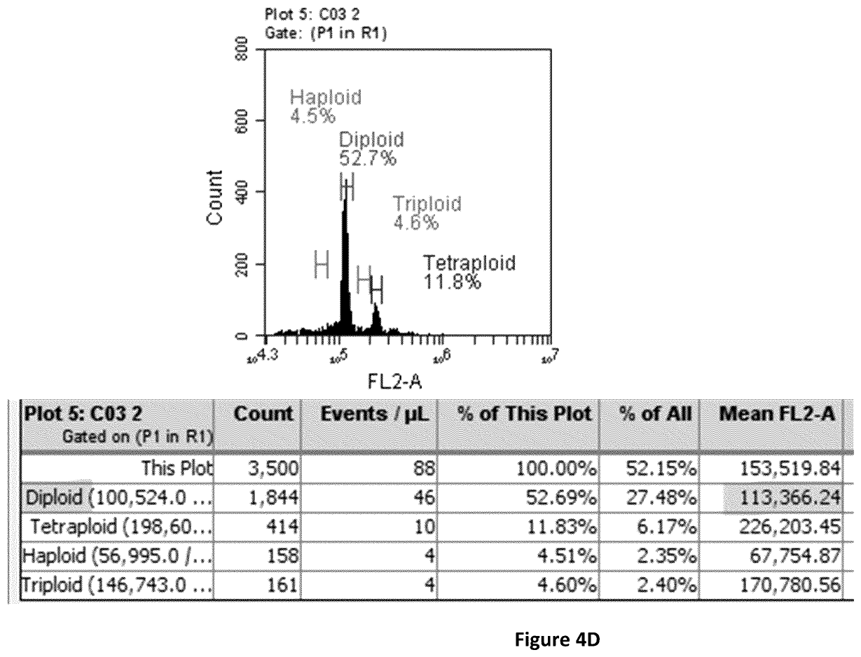

FIG. 4A shows exemplary leaves from the cultivar “Cryptic Lineage Legacy” either (left) in a haploid (n) state, or (middle) mixaploid (n+2n) state, and (right) from the diploid (2n) parent of the haploid. A “mixaploid state” refers to a condition in which a plant or organism has cells with varying ploidy levels, meaning some cells have different numbers of chromosome sets.

FIG. 4B shows exemplary flow cytometer data from the cultivar “Cryptic Lineage Legacy” in a haploid (n) state, in which approximately 59.11% of the cells counted are haploid, approximately 12.43% are diploid. The diploid cells are likely a result of naturally occurring endoreduplication or spontaneous doubling, whereas the triploid and tetraploid cell counts do not have a distinct peak apart from the background noise.

FIG. 4C shows an exemplary mixaploid (n+2n) state plot shows a ploidy of approximately 20.65% diploid, approximately 17.65% haploid, and approximately 2.63% tetraploid. The mixaploid state has a pattern distinct from the haploid and diploid, which show mostly diploid cells, which likely accounts for a mostly successful doubling, but also a stubbornly stable subset of haploid cells, which do have their own distinct peak.

FIG. 4D shows an exemplary diploid (2n) parent of the haploid shows approximately 52.69% diploid, approximately 11.83% tetraploid, which is most likely from naturally occurring endoreduplication. This diploid parent also has counts from haploid and triploid cells that do not have their own distinct peak apart from the noise.

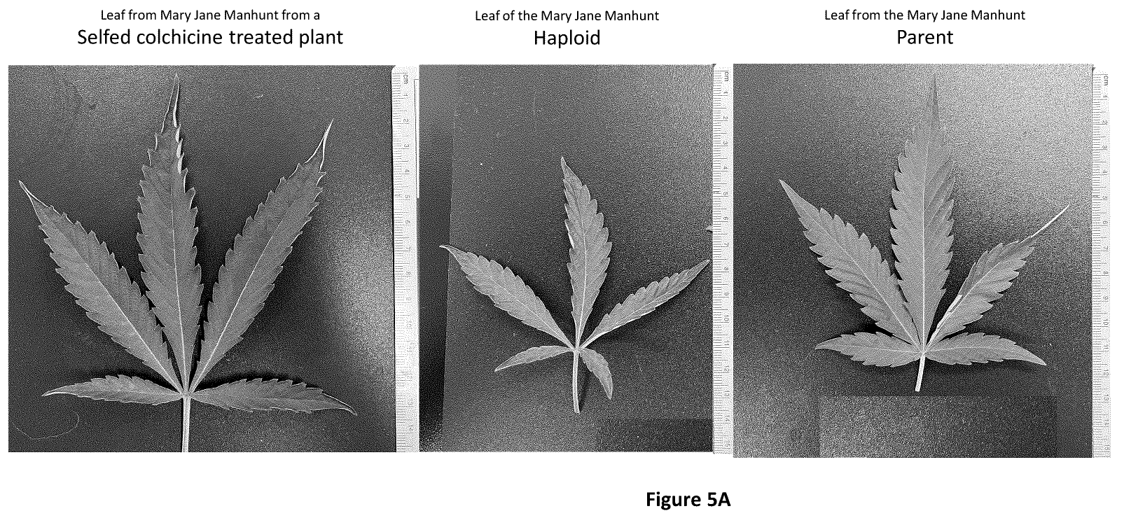

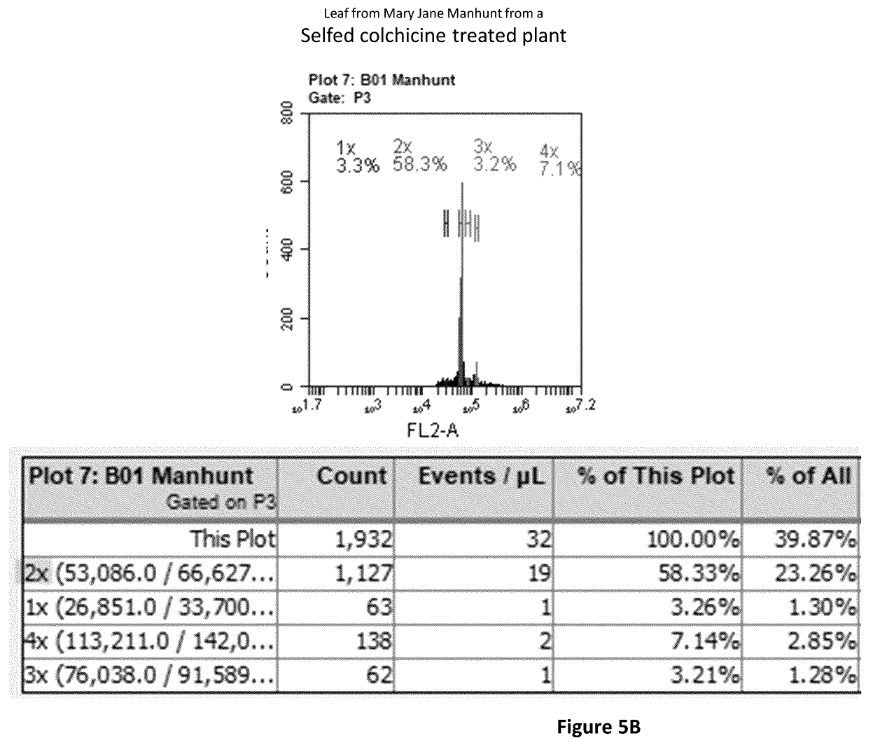

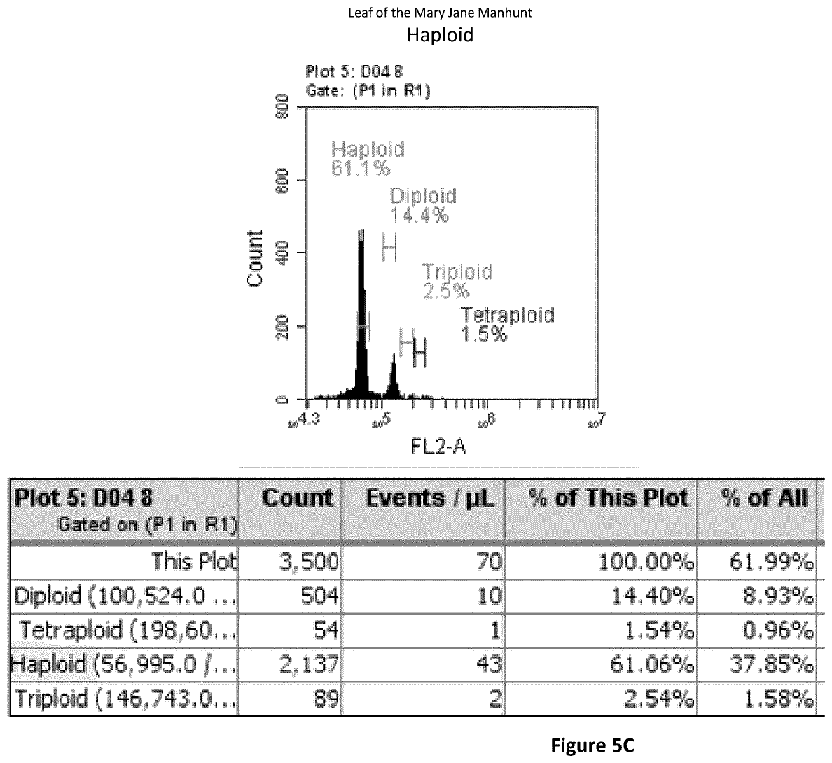

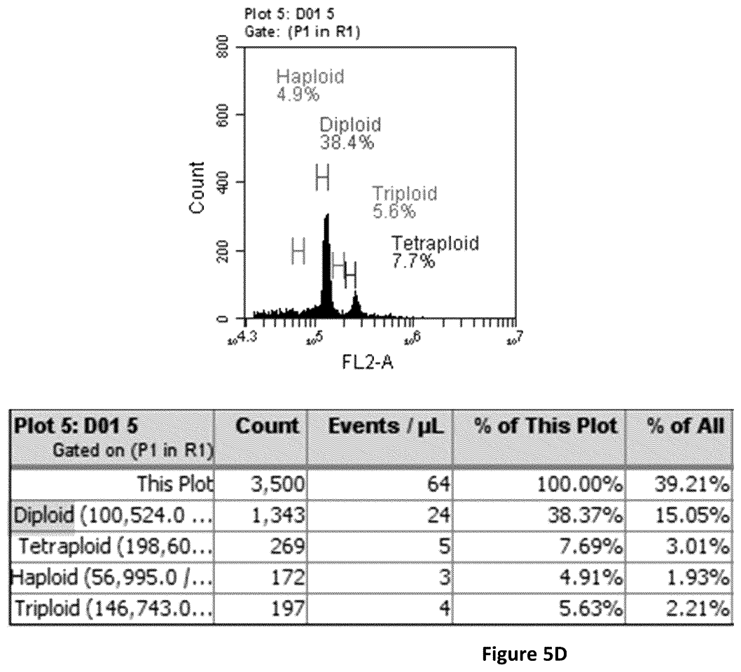

FIG. 5A shows exemplary leaves from the (left) self-fertilized doubled haploid cultivar “Mary Jane Manhunt” in a diploid (2n) state, or (middle) haploid (n) state, and (right) from the diploid (2n) parent of the haploid. The mixaploid data for the colchcine treated haploid was not gathered.

FIG. 5B shows exemplary flow cytometer data from the self-fertilized doubled haploid cultivar “Mary Jane Manhunt” in a diploid (2n) state, in which approximately 58.33% of the cells counted are diploid, approximately 7.4% are tetraploid. This self-fertilized doubled haploid cultivar also has counts from haploid and triploid cells that do not have their own distinct peak apart from the noise.

FIG. 5C shows an exemplary haploid (n) state plot shows a ploidy of approximately 61.06% haploid, approximately 14.4% diploid. The diploid cells are likely a result of naturally occurring endoreduplication or spontaneous doubling, whereas the triploid and tetraploid cell counts do not have a distinct peak apart from the background noise.

FIG. 5D show an exemplary diploid (2n) parent of the haploid shows approximately 38.37% diploid, approximately 7.69% tetraploid, which is most likely from naturally occurring endoreduplication. This diploid parent also has counts from haploid and triploid cells that do not have their own distinct peak apart from the noise.

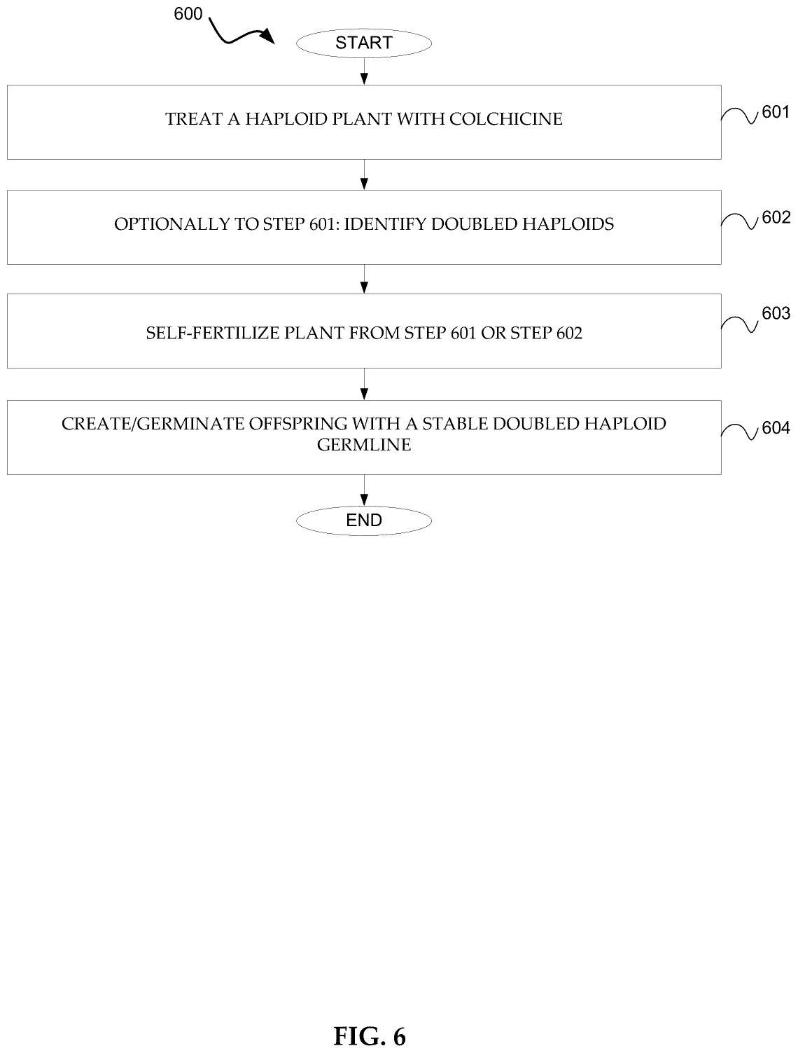

FIG. 6 shows one exemplary method 600 of stabilizing ploidy.

At step 601, a plant is treated with colchicine (or any chemical that will achieve increasing a ploidy level such as a doubling of the chromosomes with Oryzalin). In various exemplary methods, the plant is from the genus Cannabis (hemp or marijuana).

Oryzalin belongs to the dinitroaniline class of herbicides and works by inhibiting the formation of microtubules, which are essential for cell division and growth. Colchicine is a naturally occurring alkaloid derived from the plant Colchicum autumnale, commonly known as autumn crocus or meadow saffron. Colchicine works by inhibiting microtubule polymerization, which disrupts various cellular functions, including mitosis (cell division).

Micro tubule polymerization is a cellular process involving the assembly of microtubules, which are dynamic, cylindrical structures made of tubulin protein subunits. Microtubules are a critical component of the cytoskeleton, providing structural support, facilitating intracellular transport, and playing key roles in cell division and motility.

Microtubules are composed of α-tubulin and β-tubulin dimers. These dimers polymerize to form protofilaments, which then associate laterally to create the hollow, tube-like structure of a microtubule.

### Polymerization Dynamics:

Nucleation: The initial step where a small number of tubulin dimers come together to form a stable nucleus, which serves as a template for further growth.

Elongation: The addition of tubulin dimers to the growing ends of the microtubule. Microtubules have a dynamic nature, with a “plus” end that grows faster and a “minus” end that grows more slowly.

Dynamic Instability: Microtubules constantly switch between phases of growth (polymerization) and shrinkage (depolymerization), a phenomenon known as dynamic instability. This allows the cell to rapidly reorganize its cytoskeleton in response to various stimuli.

### Role of Microtubules:

Cell Division: Microtubules form the mitotic spindle, which is crucial for the segregation of chromosomes during mitosis and meiosis.

Intracellular Transport: Microtubules serve as tracks for the movement of organelles and vesicles, with motor proteins like kinesin and dynein transporting cargo along these tracks.

Cell Shape and Motility: Microtubules help maintain cell shape and enable cellular movements, including cilia and flagella beating and amoeboid movement.

### Regulation of Microtubule Dynamics:

Microtubule-Associated Proteins (MAPs): These proteins bind to microtubules and regulate their stability and dynamics. Examples include tau protein and MAP2.

GTP Hydrolysis: Tubulin dimers bind to GTP before adding to the microtubule. GTP hydrolysis to GDP after incorporation affects the stability of the microtubule, promoting dynamic instability.

### Inhibitors of Microtubule Polymerization:

Certain drugs and natural compounds can inhibit microtubule polymerization by binding to tubulin and preventing its assembly. These include:

Colchicine: Binds to tubulin, inhibiting microtubule polymerization, and is used to treat gout.

Vincristine and Vinblastine: Chemotherapy agents that disrupt microtubule dynamics, inhibiting cell division.

Taxanes (e.g., Paclitaxel): Stabilize microtubules and prevent their depolymerization, also used in cancer therapy.

At optional step 602, identify doubled haploids.

Spontaneously doubled haploids are plants that naturally undergo chromosome doubling from the haploid state to the diploid state. This process results in plants that are homozygous for all genes, meaning they carry identical alleles for every gene.

At step 603, the plant from step 601 or 602 is self-fertilized.

Self-fertilization, or selfing, in plants is a reproductive process where a plant's sperm cells (pollen) fertilize its own egg cells (ovules). This leads to the production of seeds that develop into new plants. Self-fertilization can occur in plants that possess both male (anthers) and female (stigma) reproductive structures. Self-fertilization can also occur if a clone is used to fertilize another clone of the same genetic background.

At step 604, create/germinate offspring with a stable doubled haploid germline.

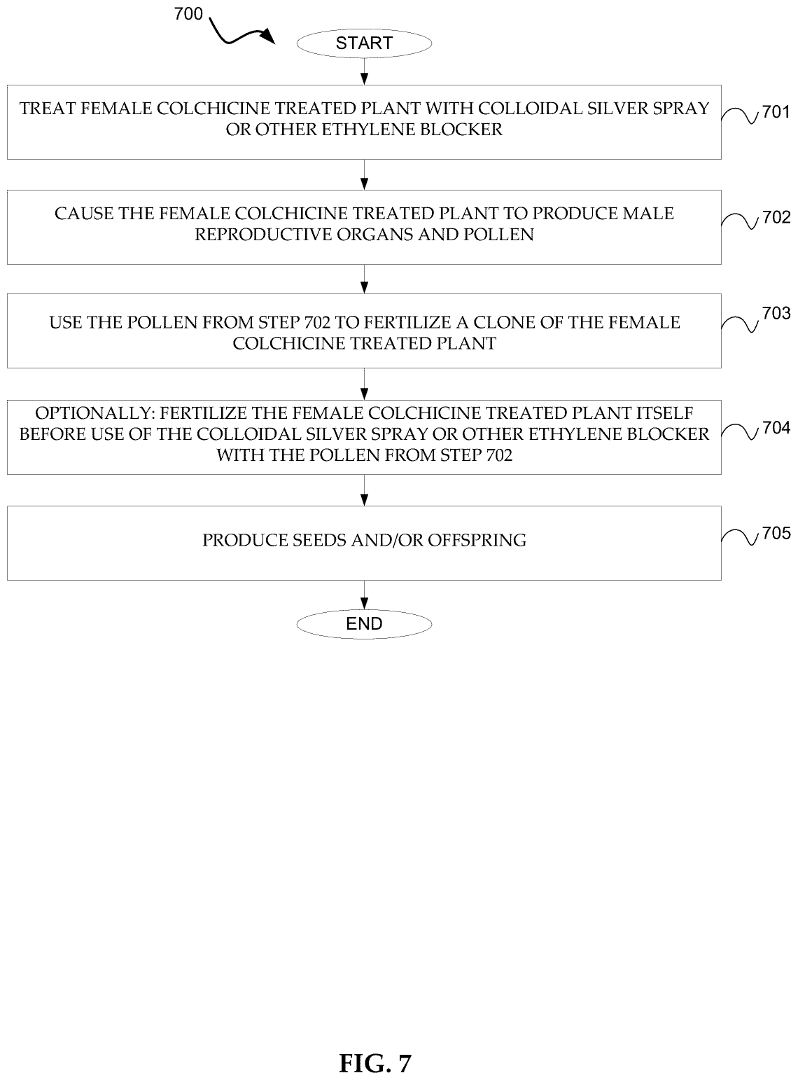

FIG. 7 shows another exemplary method 700 of stabilizing ploidy.

At step 701, a female colchicine (or any chemical that will achieve increasing a ploidy level such as a doubling of the chromosomes with Oryzalin) treated plant is treated with a colloidal silver spray or other ethylene response blocker.

Colloidal silver spray is a suspension of microscopic silver particles dispersed in water. It is often used for its purported antibacterial, antiviral, and antifungal properties. The silver particles in colloidal silver are typically between 1 and 100 nanometers in size. These particles are suspended in a liquid, usually distilled water.

An ethylene response blocker is a substance that inhibits the action or production of ethylene, a plant hormone involved in regulating various physiological processes such as fruit ripening, flower wilting, leaf abscission, and senescence. By blocking ethylene's effects, these substances can extend the shelf life of fruits and flowers, delay senescence, and manage other ethylene-related processes in plants. Ethylene response blockers prevent the physiological changes induced by ethylene by inhibiting its synthesis, perception, or signal transduction pathway.

### Types of Ethylene Response Blockers:

1-Methylcyclopropene (1-MCP): One of the most widely used ethylene blockers. It binds to the ethylene receptors in plant cells, preventing ethylene from binding and triggering its effects. It is used in the storage and transport of fruits and flowers.

Silver Thiosulfate (STS): An inhibitor of ethylene action used mainly in the floral industry to prolong the vase life of cut flowers.

Aminoethoxyvinylglycine (AVG): Inhibits ethylene biosynthesis by blocking the activity of the enzyme ACC synthase, which is critical in the ethylene production pathway.

Ethylene Antagonists: Compounds like potassium permanganate that oxidize ethylene to carbon dioxide and water, removing it from the environment around stored produce.

### Mechanism of Action:

Ethylene response blockers can work at different points in the ethylene pathway:

Biosynthesis Inhibition: Preventing the production of ethylene within the plant.

Receptor Blocking: Competing with ethylene for receptor sites, preventing it from binding and activating the signal transduction pathway.

Signal Transduction Interruption: Disrupting the downstream signaling cascade that leads to ethylene's physiological effects.

At step 702, the female colchicine (or any chemical that will achieve increasing a ploidy level such as a doubling of the chromosomes with Oryzalin) treated plant produces male reproductive organs and pollen. This is most likely caused by step 701.

At step 703, the pollen produced at step 702 is used to fertilize a clone of the female colchicine (or any chemical that will achieve increasing a ploidy level such as a doubling of the chromosomes with Oryzalin) treated plant.

A clone of a plant is a genetically identical copy of the original plant. Cloning in plants can occur naturally or be induced artificially through various horticultural techniques.

### Key Aspects of Plant Cloning:

### Natural Cloning:

Vegetative Reproduction: Many plants naturally produce clones through vegetative reproduction. This involves the growth of new plants from parts of the parent plant, such as stems, roots, or leaves.

### Artificial Cloning Techniques:

Cuttings: One of the most common methods of cloning plants is taking cuttings. A piece of the stem, leaf, or root is cut from the parent plant and placed in a suitable growing medium to develop roots and grow into a new plant.

Layering: In this technique, a branch or stem is bent to the ground and covered with soil while still attached to the parent plant. Once roots develop, the new plant is separated from the parent.

Air Layering: Inducing root formation on a branch or stem while it is still attached to the parent plant, often by wrapping it with moist moss and plastic.

Grafting and Budding: This involves joining parts of two plants together so they grow as one. A piece of a desired plant (scion) is attached to a rootstock. This technique is widely used in fruit tree propagation.

### Tissue Culture (Micropropagation):

In Vitro Cloning: This highly controlled method involves growing plant cells, tissues, or organs in a sterile environment on a nutrient culture medium. Tissue culture allows for the mass production of clones from a small amount of plant material and is used for plants that are difficult to propagate by traditional methods. Steps include:

Initiation: Explants (small pieces of plant tissue) are taken from the parent plant and sterilized.

Multiplication: Explants are placed on a growth medium containing nutrients and hormones to stimulate cell division and the formation of new shoots.

Rooting: Shoots are transferred to a rooting medium to develop roots.

Acclimatization: The new plantlets are gradually acclimatized to normal growing conditions.

At optional step 704, the pollen produced at step 702 is used to fertilize the female colchicine treated (or any chemical that will achieve increasing a ploidy level such as a doubling of the chromosomes with Oryzalin) plant itself before use of the colloidal silver spray or other ethylene blocker.

At step 705, seeds and/or offspring are produced.

### Example One: Female Sex Reversal in Cannabis

Short-day (long-night) Cannabis plants are grown for at least 4 weeks under 18-hour day, 6-hour night light cycle and were thoroughly sprayed in the dark with 2.7 mM AgNO3 and 11 mM Sodium thiosulfate solution. A 2.7 mM solution of AgNO₃ refers to a concentration of silver nitrate (AgNO₃) in a solution where there are 2.7 millimoles of AgNO₃ per liter of solution. An 11 mM sodium thiosulfate solution refers to a solution where the concentration of sodium thiosulfate (Na₂S₂O₃) is 11 millimoles per liter. These plants were left in the dark for 12 hours with adequate airflow to allow the spray to dry on the plant. These plants were then grown under a 12-hour day, 12-hour night light cycle and sprayed at the beginning of the night cycle every 3 days for a total of 4 treatments. Pollen is usually ready 6 weeks after the first treatment. Pollen from the reversed plant is then applied on stigma of a female plant that has been grown in a 12-hour day and 12-hour night light cycle for 3 weeks.

### WORKS CITED

De Rybel, Bert, et al. “Plant vascular development: from early specification to differentiation.” Nature reviews Molecular cell biology 17.1 (2016): 30-40.
Heidstra, R., Sabatini, S. Plant and animal stem cells: similar yet different. Nat Rev Mol Cell Biol 15, 301-312 (2014).
Meyerowitz, Elliot M. “Genetic control of cell division patterns in developing plants.” Cell 88.3 (1997): 299-308.
Sakhanokho, H. F., Christopher Cheatham, and J. C. Pounder. “Evaluation of techniques to induce polyploid daylilies.” Southern Nursery Association Proceedings. Vol. 49. 2004.
Satina, Sophie, Albert Francis Blakeslee, and Amos G. Avery. “Demonstration of the three germ layers in the shoot apex of Datura by means of induced polyploidy in periclinal chimeras.” American Journal of Botany (1940): 895-905.

1. A method of stabilizing ploidy comprising:
doubling a plant's ploidy;
self-fertilizing the doubled plant; and
creating an offspring with a germline ploidy of a desired doubled ploidy targeted when doubling the plant's ploidy.

2. A method of stabilizing ploidy comprising:
self-fertilizing a haploid plant; and
creating an offspring plant with a germline ploidy that is diploid.

3. A method of stabilizing ploidy comprising:
treating a female microtubule inhibitor treated plant with an ethylene blocker;
producing male reproductive organs and pollen by the female microtubule inhibitor treated plant;
fertilizing a clone of the female microtubule inhibitor treated plant with the pollen; and
producing seeds and/or offspring.

4. The method of claim 1, further comprising the plant being from a Cannabis genus.

5. The method of claim 2, further comprising the plant being from a Cannabis genus.

6. The method of claim 3, further comprising the plant being from a Cannabis genus.

7. The method of claim 4, the Cannabis being marijuana.

8. The method of claim 5, the Cannabis being marijuana.

9. The method of claim 6, the Cannabis being marijuana.

10. The method of claim 4, the Cannabis being hemp.

11. The method of claim 5, the Cannabis being hemp.

12. The method of claim 6, the Cannabis being hemp.

13. The method of claim 3, further comprising fertilizing the female microtubule inhibitor treated plant itself with the pollen that it produces.

14. The method of claim 1, further comprising identifying a plant with spontaneously doubled haploids.

15. The method of claim 2, further comprising the offspring plant with the germline ploidy being 100% diploid.
